(19) United States
(12) Patent Application Publication
(10) Pub. No.: US 2010/0040671 A1
(43) Pub. Date: Feb. 18, 2010

(54) Intravaginal Devices With a Rigid Support, Methods of Making, and Uses Thereof

(76) Inventors: Salah U. AHMED, New City, NY (US); Jiaxiang Tsao, Nanuet, NY (US)

Correspondence Address: Sterne, Kessler, Goldstein & Fox PLLC, 1100 New York Avenue, NW, Washington, DC, 20005, US

(21) Appl. No.: 12/539,568

(22) Filed: Aug. 11, 2009

Related U.S. Application Data
(60) Provisional application No. 61/088,314, filed on Aug. 12, 2008.

(51) Int. Cl. A61F 6/14; B28B 1/14; A61F 6/06

(52) U.S. Cl. 424/432; 264/299; 128/834

(57) ABSTRACT

The present invention relates to intravaginal devices having a rigid support, methods of making, and uses thereof. The devices comprise (a) a rigid support having a Shore A Hardness of at least about 20 and a tensile strength of at least about 1 MPa, (b) a matrix, and (c) an active agent dispersed in the matrix, wherein the support and the matrix are adjacent and wherein the device is annular.

This application claims benefit of the filing date of U.S. Application No. 61/088,314, filed Aug. 12, 2008, which is hereby incorporated by reference in its entirety.

### FIELD OF THE INVENTION

The present invention relates to intravaginal devices having a rigid support, methods of making the intravaginal devices, and methods of use thereof. The intravaginal devices comprise (a) a rigid support having a Shore A Hardness of at least about 20 and a tensile strength of at least about 1 MPa, (b) a matrix, and (c) an active agent dispersed in the matrix, wherein the support and the matrix are adjacent and wherein the device is annular.

### BACKGROUND OF THE INVENTION

Intravaginal delivery has been used previously as a method of active agent administration. In some instances intravaginal delivery provides good adsorption of active agents while avoiding the first-pass effect in the liver. As a result, intravaginal delivery has been considered an efficacious method for administering many types of active agents. Intravaginally administered active agents can directly diffuse through the vaginal tissues to provide a local effect or a systemic effect, thereby treating numerous conditions within and outside the vaginal and/or urogenital tract, such as hormonal dysfunctions, inflammation, infection, pain, and incontinence. Several methods of intravaginal drug delivery exist in the art, such as for example, the use of intravaginal rings, intrauterine devices, and intravaginal pessaries (see e.g., U.S. Pat. Nos. 4,823,814; 4,607,630; 4,553,972; 4,286,587; and 4,249,531). Of these methods, intravaginal rings provide a versatile, comfortable method for delivering active agents which can be readily inserted and removed.

There is a need in the art for improved intravaginal devices capable of delivering active agents to the vaginal and/or urogenital tract, with the devices having increased physical integrity, safety, and comfort.

### BRIEF SUMMARY OF THE INVENTION

The present invention is directed to an intravaginal device having a rigid support and a matrix. The present invention is also directed to an intravaginal device for delivering an active agent to a subject, wherein the intravaginal device can further have an active agent dispersed in the matrix. In some embodiments, the present invention is directed to an intravaginal device comprising (a) a rigid support having a Shore A Hardness of at least about 20 and a tensile strength of at least about 1 MPa, (b) a matrix, and (c) an active agent dispersed in the matrix, wherein the support and the matrix are adjacent and wherein the device is annular.

The present invention is directed to a method of making an intravaginal device having a rigid support and a matrix, the method comprising (i) placing the support in a mold, wherein the support has a Shore A Hardness of at least about 20 and a tensile strength of at least about 1 MPa, and (ii) adding the matrix to the mold, wherein the matrix has an active agent dispersed therein, wherein the support and the matrix are adjacent and wherein the device is annular. The present invention is also directed to a device made by this method.

The present invention is directed to a method of contraception, the method comprising administering to a female the intravaginal device of the present invention. The present invention is also directed to a method of hormone replacement therapy, the method comprising administering to a female the intravaginal device of the present invention.

In some embodiments, the support has a Shore A Hardness of about 20 to about 80. In some embodiments, the support has a tensile strength of about 1 MPa to about 100 MPa. In some embodiments, the support has a Shore A Hardness of about 20 to about 80 and a tensile strength of about 1 MPa to about 100 MPa.

In some embodiments, the support comprises a metal, an alloy, a plastic, composites thereof, or combinations thereof. In some embodiments, the plastic comprises a thermoplastic or a thermoset plastic. In some embodiments, the plastic comprises a polyalkylene, a polystyrene, a polysiloxane, a polyvinyl acetate, a polyvinyl chloride, a polyester, a polyurethane, an acrylic, a nylon, a dacron, a teflon, or combinations thereof. In some embodiments, the polyalkylene is a high density polyethylene, a high density polypropylene, a high density polybutylene, or combinations thereof. In some embodiments, the polyalkylene is a high density polyethylene.

In some embodiments, the matrix comprises a polysiloxane, a polyalkylene, a polystyrene, a polyvinyl acetate, a polyvinyl chloride, a polyester, a polyurethane, an acrylic, a nylon, a dacron, a teflon, or combinations thereof. In some embodiments, the matrix is a polysiloxane, an ethylene-vinylacetate copolymer, or combinations thereof.

In some embodiments, the matrix encompasses at least 50% of the surface area of the support. In some embodiments, the matrix encompasses at least 95% of the surface area of the support. In some embodiments, the device comprises more than one matrix.

In some embodiments, the active agent is a steroid hormone, an anticholinergic, an anesthetic, combinations thereof, or derivatives thereof. In some embodiments, the active agent is progesterone, estrogen, oxybutynin, lidocaine, danazol, etonogestrel, ethinyl estradiol, or combinations thereof.

In some embodiments, the active agent is released from the device at an average rate of about 0.01 mg to about 10 mg per 24 hours in situ. In some embodiments, the active agent is released from the device at an average rate of about 1 mg to about 100 mg per 24 hours in situ. In some embodiments, the active agent is supersaturated in the matrix at 25° C.

In some embodiments, the device is a vaginal ring. In some embodiments, the device has an outer diameter of about 40 mm to about 70 mm. In some embodiments, the device has an inner diameter of about 10 mm to about 60 mm. In some embodiments, the device has a cross-sectional diameter of about 1 mm to about 8 mm. In some embodiments, the support has a cross-sectional diameter of about 0.5 mm to about 4 mm. In some embodiments, the device has a surface area of about 800 mm² to about 2000 mm². In some embodiments, the device further comprises an outer sheath.

### BRIEF DESCRIPTION OF THE FIGURES

FIG. 1 depicts a top-down view and a cross-sectional view of a vaginal ring having a silicone polymer matrix (a), wherein (b) represents the outer diameter and (c) represents the cross-sectional diameter.

FIG. 2 depicts a top-down view and a cross-sectional view of a vaginal ring having a solid support, wherein (b) represents the silicone polymer matrix and (c) represents a rigid support of medical-grade HDPE.

FIG. 3 depicts a top-down view and a cross-sectional view of a vaginal ring having a solid support and two matrices, wherein (A) represents a rigid support of medical-grade HDPE, (B) represents a first polymer matrix and (C) represents a second polymer matrix encompassing the first matrix.

FIG. 4 depicts a top-down view of a vaginal ring having a solid support and two matrices constituting separate segments of the ring, wherein (A) represents a rigid support of medical-grade HDPE, (B) represents a first polymer matrix encompassing a portion of the support and (C) represents a second polymer matrix encompassing a different portion of the support.

### DETAILED DESCRIPTION OF THE INVENTION

The present invention relates to intravaginal devices having a rigid support, methods of making the intravaginal devices, and methods of use thereof.

Throughout the present disclosure, all expressions of percentage, ratio, and the like are “by weight” unless otherwise indicated. As used herein, “by weight” is synonymous with the term “by mass,” and indicates that a ratio or percentage defined herein is done according to weight rather than volume, thickness, or some other measure.

As used herein, the term “about,” when used in conjunction with a percentage or other numerical amount, means plus or minus 10% of that percentage or other numerical amount. For example, the term “about 80%,” would encompass 80% plus or minus 8%.

### Intravaginal Devices

The present invention is directed to an intravaginal device having a rigid support and a matrix. The present invention is also directed to an intravaginal device for delivering an active agent to a subject, wherein the intravaginal device can further have an active agent dispersed in the matrix. The present invention is further directed to an intravaginal device comprising (a) a rigid support having a Shore A Hardness of at least about 20 and a tensile strength of at least about 1 MPa, (b) a matrix, and (c) an active agent dispersed in the matrix, wherein the support and the matrix are adjacent and wherein the device is annular.

As used herein, an “intravaginal device” refers to an object that provides for administration or application of an active agent to the vaginal and/or urogenital tract of a subject, including, e.g., the vagina, cervix, or uterus of a female.

In some embodiments, the device is annular in shape. As used herein, “annular” refers to a shape of, relating to, or forming a ring. Annular shapes suitable for use with the present invention include a ring, an oval, an ellipse, a toroid, and the like. In some embodiments, the intravaginal device of the present invention is a vaginal ring.

As used herein, “adjacent” refers to being in close proximity and may or may not imply direct contact between materials. In some embodiments, the support and the matrix are in contact. In some embodiments, the support and the matrix have an intervening material in between, e.g., a film, layer, barrier, sheath, etc.

As used herein, a “rigid support” refers to a portion of the device that adds physical integrity to the intravaginal device. In some embodiments, the support is resistant to compression and deformation. In some embodiments, “rigid” can be defined in terms of the hardness and tensile strength of the material comprising the support.

The intravaginal device comprises a rigid support having a specified hardness. As used herein, “hardness” refers to a measurement of the resistance of a material to compression, indentation, scratching, and deformation that correlates well with mechanical strength, rigidity, and resistance to abrasion. For example, a known preferred hardness testing method for elastomers and plastics is the Shore Hardness test. The “Shore Hardness” value of a plastic material sample can be measured with an apparatus known as a Durometer and consequently is also known as Durometer hardness. The hardness value can be determined by penetration of a Durometer indenter foot into a sample. If the indenter completely penetrates the sample, a reading of 0 is obtained, and if no penetration occurs, a reading of 100 results. Durometers can measure hardness in different ways, depending on the spring force and indenter geometry used. Several Shore Hardness scales can be used, e.g., a Shore A scale and a Shore D scale. The “Shore A Hardness” relates to the relative hardness of elastic materials such as rubber or plastics and can be determined using various methods and equipment known in the art. In some embodiments, a Shore A Durometer is used. In some embodiments, Shore A Hardness can be determined in accordance with the standard test method recited in ASTM D-2240-91, using a Shore “Type A” Durometer having a maximum indicator (available from Shore Instrument and Manufacturing Company, Inc., Freeport, N.Y.).

The rigid support on the intravaginal device of the present invention can have various Shore A Hardness values. In some embodiments, the rigid support has a Shore A Hardness of at least about 20, or about 20 to about 200, or about 30 to about 150, or about 40 to about 100, or about 50 to about 80, or about 20 to about 60, or about 60 to about 120, or about 70 to about 120, or about 80 to about 120.

In some embodiments, the rigid support has various tensile strengths. As used herein, “tensile strength” refers to the ratio of the amount of axially applied force required to break or rupture a material to the cross-sectional area of the material. As used herein, the term “breaking force” is the maximum force which the specimen can bear before breaking. Tensile strength, then, represents the stress on a material at the time of rupture and can be calculated by dividing the breaking force by the cross-sectional area. Tensile strength is measured in units of force per unit area, e.g., Newtons per square meter (N/m²) or pascals (Pa). Tensile strength values can be determined using methods and equipment known in the art. For example, tensile strength can be determined in accordance with the standard test method recited in ASTM D638-98 by using, e.g., a Tensile Testing Machine (available from United Testing Systems, Inc., Huntington Beach, Calif.).

An intravaginal device with a rigid support having various tensile strengths can be used in the present invention. In some embodiments, a rigid support has a tensile strength of at least about 1 MPa, or about 1 MPa to about 200 MPa, or about 5 MPa to about 150 MPa, or about 10 MPa to about 100 MPa, or about 20 MPa to about 75 MPa, or about 25 MPa to about 50 MPa, or about 1 MPa to about 25 MPa, or about 15 MPa to about 100 MPa, or about 35 MPa to about 100 MPa, or about 50 MPa to about 100 MPa.

In some embodiments, the intravaginal device of the present invention has a support with a Shore A Hardness of at least about 20 and a tensile strength of at least about 1 MPa, or with a Shore A Hardness of about 20 to about 200 and a tensile strength of about 1 MPa to about 200 MPa, or with a Shore A Hardness of about 20 to about 100 and a tensile strength of about 1 MPa to about 150 MPa, or with a Shore A Hardness of about 20 to about 80 and a tensile strength of about 1 MPa to about 100 MPa, or with a Shore A Hardness of about 60 to about 120 and a tensile strength of about 15 MPa to about 100 MPa, or with a Shore A Hardness of about 70 to about 120 and a tensile strength of about 35 MPa to about 100 MPa, or with a Shore A Hardness of about 80 to about 120 and a tensile strength of about 50 MPa to about 100 MPa.

The intravaginal device of the present invention can be flexible. As used herein, “flexible” refers to the ability of a solid or semi-solid to bend or withstand stress and strain without being damaged or broken. For example, the device of the present invention can be deformed or flexed, such as, for example, using finger pressure (e.g., applying pressure from opposite external sides of the device using the fingers), and upon removal of the pressure, return to its original shape. The flexible properties of the intravaginal device of the present invention are useful for enhancing user comfort, and also for ease of administration to the vaginal tract and/or removal of the device from the vaginal tract.

Intravaginal devices having a rigid support have increased physical integrity relative to intravaginal devices without a rigid support. In some embodiments, the increased physical integrity can achieve a higher safety profile while administering the active agent due to reduced instances of breaks, tears, ruptures and leakages of active agent from the intravaginal device. In some embodiments, the increased physical integrity can achieve a more consistent release profile while administering the active agent.

Materials used in the intravaginal device of the present invention are suitable for placement in the vaginal tract, i.e., they are nontoxic and can further be non-absorbable in the subject. In some embodiments, the materials are compatible with an active agent. In some embodiments, the materials can be capable of being suitably shaped for intravaginal administration.

In some embodiments, the support of the present invention can be made from any material which increases the physical integrity of the intravaginal device. In some embodiments, the support comprises any material which adds rigidity to the intravaginal device. In some embodiments, e.g., the support can be a metal, an alloy, a plastic, composites thereof, or combinations thereof. One of skill in the art will recognize that various factors can effect the rigidity of a material, e.g., the size of the material, shape of the material, amount of the material, thickness of the material, etc.

As used herein, an “alloy” refers to a substance composed of two or more metals or of a metal and a nonmetal. Suitable metals or alloys include, but are not limited to, unalloyed and alloyed titanium, molybdenum, chromium, cobalt, tungsten, aluminum, niobium, manganese, iron or vanadium, combinations thereof, or oxides thereof. In some embodiments, the alloy is a titanium alloy, a cobalt alloy, or a stainless steel alloy.

As used herein, a “plastic” refers to any of numerous organic synthetic or processed thermoplastic or thermosetting polymers. Suitable plastic polymers include, but are not limited to the polyhydroxy acids poly(lactic acid) (PLA), poly(glycolic acid) (PGA), poly(lactides-co-glycolides) (PLGA), poly(DL-lactide-ε-caprolactone), and polyurethane polymers.

In some embodiments, the plastic of the rigid support can be a thermoplastic or a thermoset plastic. As used herein, a “thermoplastic polymer” is a polymer capable of being softened by heating and hardened by cooling through a temperature range characteristic of the polymer. In the softened state, thermoplastic polymers can be shaped by flow, molding or extrusion. Suitable thermoplastic polymer materials include, but are not limited to, a thermoplastic olefin blend, a crosslinked polymer, a copolymer, a block copolymer, and combinations thereof. In some embodiments, thermoplastic polymers suitable for use in the present invention include, but are not limited to, nylon, polyethylene, polypropylene, polyvinyl acetate (PVA), polyvinyl chloride (PVC), polystyrene, teflon, acrylic, ethylene-vinylacetate copolymers, and styrene-butadiene-styrene copolymers.

Thermoplastic elastomeric polymers can be used in the rigid support of the present invention. As used herein, “thermoplastic elastomeric polymers” are any materials that possess elasticity and resilience at ambient temperatures, without the need for vulcanization to develop rubberlike elasticity. Suitable thermoplastic elastomeric materials include, but are not limited to, polyurethanes, polyesters, polyalkylenes, and combinations thereof. In some embodiments, the thermoplastic elastomeric material can be, but is not limited to, a thermoplastic olefin blend, an ionomer, a block copolymer, or combinations thereof. In some embodiments, the thermoplastic elastomeric material is a styrene-ethylene-butylene modified block copolymer.

In some embodiments of the invention, the rigid support comprises a thermoset plastic polymer. As used herein, a “thermoset plastic polymer” is any material that is shaped irreversibly under the influence of heat through the formation of a covalently linked, thermally stable network. Suitable thermoset plastic materials include, but are not limited to, crosslinked polymers, copolymers, block copolymers, and combinations thereof.

In some embodiments of the invention, the polymer material is an elastomer, e.g., a thermosetting elastomer, including, e.g., a silicone co-polymer (thermosetting type). For example, the intravaginal device of the present invention can be produced using silicone polymers which may include various catalysts or cross-linking agents. Such silicone compounds, catalysts and crosslinking agents are known in the art, see e.g., U.S. Pat. No. 4,888,074. A silicone composition can include any organo-silicone compound capable of crosslinking, with or without the presence of crosslinking agents.

In some embodiments, the plastic can be a polyalkylene, a polystyrene, a polysiloxane, a polyvinyl acetate, a polyvinyl chloride, a polyester, a polyurethane, an acrylic, a nylon, a dacron, a teflon, or combinations thereof.

In some embodiments, the polyalkylene can be a high density polyethylene, a high density polypropylene, a high density polybutylene, or combinations thereof. In some embodiments, the polyalkylene of the support can be a high density polyethylene. As used herein, “high density polyethylene” (HDPE) refers to a material comprising a substantially linear, semi-crystalline, polymer of ethylene. In some embodiments, the HDPE is a homopolymer. In some embodiments, the HDPE further comprises a comonomer. As used herein, the term “high density” refers to a material possessing a density of from about 0.8 to about 10 g/cm³. In some embodiments, the density of the rigid support of the device of the present invention can be from 0.8 to 10 g/cm³, from 0.8 to 8 g/cm³, from 0.8 to 6 g/cm³, from 0.8 to 4 g/cm³, from 0.8 to 2 g/cm³, from 1 to 8 g/cm³, from 1 to 4 g/cm³, from 1 to 2 g/cm³, or from 2 to 6 g/cm³. In some embodiments, the density of the rigid support of the device of the present invention can be about 0.85 g/cm³, about 0.88 g/cm³, about 0.90 g/cm³, about 0.92 g/cm³, about 0.94 g/cm³, about 0.96 g/cm³, or about 0.98 g/cm³. In some embodiments, the HDPE can be MEDPOR® Biomaterial, obtained from Porex, Newnan, Ga. The density measurement can be determined in accordance with the standard test method recited in ASTM D1505.

In some embodiments, the intravaginal device of the present invention comprises a matrix. As used herein, a “matrix” refers to a solid, semi-solid, or gel medium compatible with and suitable for dispersion of an active agent. In some embodiments, the active agent is homogeneously dispersed in the matrix. As used herein, “homogeneous” refers to a composition, e.g., the intravaginal device matrix, that has a substantially uniform distribution of ingredients throughout (i.e., an intravaginal device matrix of the present invention does not have a composition gradient, or a multi-laminate structure).

In some embodiments, the active agent is heterogeneously dispersed in the matrix. As used herein, “heterogeneous” refers to a composition, e.g., the intravaginal device matrix, that does not have a substantially uniform distribution of ingredients throughout (i.e., an intravaginal device matrix of the present invention can have a composition gradient, or a multi-laminate structure). Thus, a “heterogeneous mixture” refers to a composition of two or more ingredients, in which the ingredients are not substantially uniformly distributed (i.e., there can be segments, regions, or areas of the mixture with substantially differing amounts of any of the ingredients).

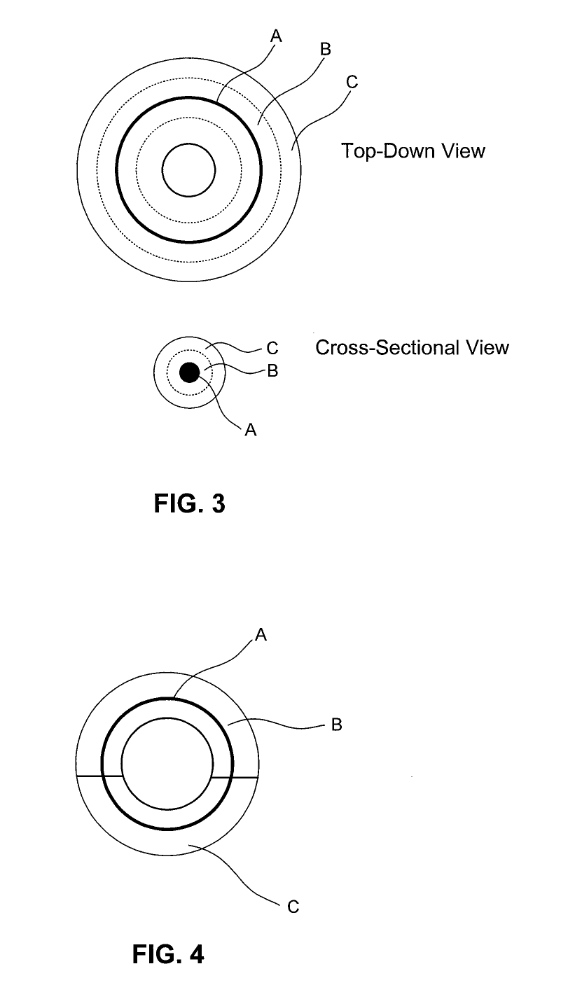

In some embodiments, the active agent does not interact or form complexes with the matrix. In some embodiments, the matrix can be chosen due to its mechanical and physical properties (e.g., solubility of an active agent in the material). In some embodiments, the device comprises more than one matrix. E.g., in some embodiments, the device comprises two, three or four matrices. In some embodiments, the second matrix encompasses the first matrix. See, e.g., FIG. 3. In some embodiments, the intravaginal device is annular in shape, and the first matrix and the second matrix constitute separate segments of the annular device. See, e.g., FIG. 4. In some embodiments, when two or more matrices are present, an active agent is in each matrix, or optionally in only one matrix.

In some embodiments, the matrix is an elastomer. As used herein, an “elastomer” refers to an amorphous polymer network formed when a polymer or a mixture of polymers undergo cross-linking. Each polymer is comprised of monomeric units, which are linked together to form the polymer. The monomeric units can comprise carbon, hydrogen, oxygen, silicon, halogen and combinations thereof.

In some embodiments, the matrix comprises a polysiloxane, a polyalkylene, a polystyrene, a polyvinyl acetate, a polyvinyl chloride, a polyester, a polyurethane, an acrylic, a nylon, a dacron, a teflon, or combinations thereof.

In some embodiments, the matrix is a polysiloxane, an ethylene-vinylacetate copolymer, or combinations thereof.

As used herein, a “polysiloxane” refers to any of various compounds containing alternate silicon and oxygen atoms in either a linear or cyclic arrangement usually with one or two organic groups attached to each silicon atom. For example, polysiloxanes include substituted polysiloxanes, and diorganopolysiloxanes such as diarylpolysiloxanes and dialkylpolysiloxanes. In some embodiments, the polysiloxane comprises a siloxane unit as illustrated in Formula I,

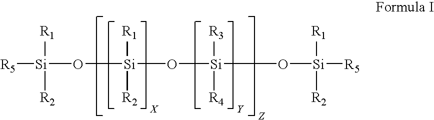

wherein X can be 1 to 200, Y can be 1 to 200 and Z can be 1 to 300, and wherein R₁, R₂, R₃, R₄ and R₅ can be independently selected from the group consisting of (C₁₋₆)alkyl, amino(C₁₋₆)alkyl, hydroxy(C₁₋₆)alkyl, haloalkyl, cyano(C₁₋₆)alkyl, thio(C₁₋₆)alkyl, carboxy(C₁₋₆)alkyl, aryl(C₁₋₆)alkyl, (C₁₋₆)alkoxy(C₁₋₆)alkyl, (C₂₋₆)alkenyl, amino(C₃₋₁₀)alkenyl, hydroxy(C₃₋₁₀)alkenyl, halo(C₂₋₆)alkenyl, cyano(C₂₋₆)alkenyl, thio(C₃₋₁₀)alkenyl, carboxy(C₃₋₁₀)alkenyl, aryl(C₂₋₆)alkenyl, (C₂₋₆)alkynyl, (C₁₋₆)heteroalkyl, (C₂₋₆)heteroalkenyl, (C₂₋₆)heteroalkynyl, (C₁₋₆)alkoxy, (C₃₋₁₀)alkenyloxy, (C₁₋₆)alkylenedioxy, amino(C₂₋₆)alkoxy, hydroxy(C₂₋₆)alkoxy, halo(C₁₋₆)alkoxy, cyano(C₁₋₆)alkoxy, thio(C₁₋₆)alkoxy, carboxy(C₂₋₆)alkoxy, aryl(C₁₋₆)alkoxy, (C₁₋₆)alkoxy(C₂₋₆)alkoxy, halo(C₁₋₆)alkoxy(C₂₋₆)alkoxy, mono(C₁₋₆)alkylamino, di(C₁₋₆)alkylamino, (C₁₋₆)alkylcarbonylamino, (C₂₋₆)alkenylcarbonylamino, (C₆₋₁₄)arylcarbonylamino, (C₁₋₆)alkoxycarbonylamino, (C₆₋₁₀)aryloxycarbonylamino, (C₁₋₆)alkylcarbonyl, (C₂₋₆)alkenylcarbonyl, (C₆₋₁₀)arylcarbonyl, (C₁₋₆)alkoxycarbonyl, (C₆₋₁₄)aryloxycarbonyl, (C₁₋₆)alkylsulfonylamino, (C₂₋₆)alkenylsulfonylamino and (C₆₋₁₄)arylsulfonylamino. In some embodiments, at least one of R₁, R₂, R₃ or R₄ is a haloalkyl.

In some embodiments, at least one of R₁-R₄ can be a mono-haloalkyl, a di-haloalkyl or a tri-haloalkyl. In some embodiments, the haloalkyl can be a bromoalkyl, chloroalkyl, fluoroalkyl or iodoalkyl. In some embodiments, the haloalkyl is a trifluoroalkyl. In some embodiments, the haloalkyl is a trifluoroethyl, trifluoropropyl or trifluorobutyl. In some embodiments, the haloalkyl is a difluoroethyl, difluoropropyl or difluorobutyl.

In some embodiments, X is 1 to 90, 10 to 80 or 20 to 70. In some embodiments, X is 1 to 10, 1 to 5 or 1 to 3. In some embodiments, Y is 1 to 90, 10 to 80 or 20 to 70. In some embodiments, Y is 1 to 10, 1 to 5 or 1 to 3. In some embodiments, Z is 10 to 250, 50 to 200 or 75 to 150. As one of skill in the art would recognize, the values of X and Y could vary in each Z subunit. Thus, e.g., X could be 3 and Y could be 4 in a first Z subunit, and then X could be 10 and Y could be 2 in a second Z subunit.

In some embodiments, R₁ is a trifluoropropyl; R₂, R₃, and R₄ are independently C₁-C₃ alkyl; R₅ is vinyl; X is 1 to 2; Y is 1 to 2; and Z is 100 to 200. In some embodiments, the polysiloxane of the present invention is trifluoropropyl methyldimethyl polysiloxane.

In some embodiments, the rigid support can be on the inside perimeter or outside perimeter of the device. In some embodiments, the matrix encompasses at least 50% of the surface area of the support. In some embodiments, the matrix encompasses at least 60% of the surface area of the support. In some embodiments, the matrix encompasses at least 70% of the surface area of the support. In some embodiments, the matrix encompasses at least 80% of the surface area of the support. In some embodiments, the matrix encompasses at least 90% of the surface area of the support. In some embodiments, the matrix encompasses at least 95% of the surface area of the support. In some embodiments, the rigid support is completely encompassed by the matrix, i.e., the matrix encompasses about 100% of the surface area of the support.

As used herein, the “encompassed surface area” refers to the area of the matrix that surrounds the support, e.g., if 70% of the surface area of the support was encompassed, then 30% of the surface area of the support would be exposed to the external environment, e.g., to the vagina, cervix, or uterus of a female.

As used herein, an “active agent” refers to a drug, protein, hormone, vitamin, nutritional supplement, or any other substance intended for use in the treatment, mitigation, cure or prevention of a disease or any other medical condition. In some embodiments, an active agent can be administered to a subject to treat a condition or a symptom thereof in a subject. For example, in some embodiments, an active agent is a component in a medicinal compound administered to treat one or more conditions, or the symptoms thereof, in a subject.

The intravaginal device of the present invention can be used to deliver one or more active agents. In some embodiments, the active agent is dispersed in the matrix. In some embodiments, the active agent is dispersed in the rigid support. In some embodiments, the active agent is dispersed in the matrix and the rigid support.

Active agents suitable for use with the present invention comprise active agents that have a localized effect, as well as systemically acting active agents that act at a point remote from the vaginal or urogenital tract. Active agents suitable for use with the present invention include, but are not limited to, an analgesic, an anti-inflammatory agent, a hormonal agent, an anti-microbial agent, an anesthetic, an anti-osteoporosis agent, an anticholinergic agent, a steroid hormone, an enzyme, and combinations thereof.

In some embodiments, the active agent is a steroid hormone, an anticholinergic, an anesthetic, combinations thereof, or derivatives thereof. A steroid hormone can include, for example, an estrogen, a progestin, a progesterone, a testosterone, derivatives thereof, or combinations thereof.

As used herein, an “estrogen” refers to any of various natural or synthetic compounds that stimulate the development of female secondary sex characteristics and promote the growth and maintenance of the female reproductive system, or any other compound that mimics the physiological effect of natural estrogens. Estrogens suitable for use with the present invention also include compounds that can be converted to active estrogenic compounds in the uterine environment. For example, in some embodiments, a conjugated estrogen can be administered from an intravaginal device of the present invention. As used herein, the term “conjugated” refers to the sulfate ester, glucuronide ester, or mixed sulfate-glucuronide esters, of an estrogen. Estrogens suitable for use with the present invention also include pharmaceutically suitable salt forms of estrogens. In some embodiments, the salt can be a sodium, potassium, or 2-amino-2-(hydroxymethyl)-1,3-propanediol (Tris) salt. In some embodiments, an estrogen suitable for use with the present invention can be useful for Hormone Replacement Therapy (HRT) regimens. In some embodiments, an estrogen suitable for use with the present invention can be useful for the treatment of osteoporosis in a subject in need thereof. In some embodiments, an estrogen suitable for use with the present invention can be useful as a contraceptive agent.

Estrogens suitable for use in the present invention include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); agonistic anti-estrogens; and selective estrogen receptor modulators.

Prodrugs of suitable estrogens can also be used in the device of the present invention. As used herein, a “prodrug” denotes a derivative of a known direct acting drug, which derivative has enhanced delivery characteristics and therapeutic value as compared to the drug and is transformed into the active drug by an enzymatic or chemical process. Examples of estrogen prodrugs include, but are not limited to, estradiol acetate (which is converted in vivo to 17β-estradiol) and mestranol (which is converted in vivo to ethinyl estradiol).

In some embodiments, the estrogen is estradiol, estriol, mestranol, ethinyl estradiol, diethylstilbestrol, or combinations thereof.

As used herein, a “progestin” refers to a progestogen, a progestational substance, or any pharmaceutically acceptable substance in the steroid art that generally possesses progestational activity including synthetic steroids that have progestational activity. Progestins suitable for use with the present invention can be of natural or synthetic origin. Progestins generally possess a cyclo-pentanophenanthrene nucleus. In some embodiments, a progestin suitable for use with the present invention can be useful for Hormone Replacement Therapy (HRT) regimens. In some embodiments, a progestin suitable for use with the present invention can be useful as a contraceptive agent.

Progestins suitable for use in the present invention include, but are not limited to, natural and synthetic compounds having progestational activity, such as, for example, progesterone, medroxyprogesterone, medroxyprogesterone acetate, chlormadinone acetate, norethindrone, cyproterone acetate, norethindrone acetate, desogestrel, levonorgestrel, drospirenone, trimegestone, norgestrel, norgestimate, norelgestromin, etonogestrel, dienogest, gestodene, megestrol, and other natural and/or synthetic gestagens.

In some embodiments, the progestin is progesterone, etonogestrel, levonorgestrel, gestodene, norethisterone, drospirenone, or combinations thereof.

Prodrugs of suitable progestins can also be used in the intravaginal device of the present invention. Ethynodiol diacetate, which is converted in vivo to norethindrone, is an example of a progestin prodrug that can be used in the present invention. Additional examples of progestin prodrugs include, but are not limited to, norgestimate (which is converted in vivo to 17-deacetyl norgestimate, also known as norelgestromin), desogestrel (which is converted in vivo to 3-keto desogestrel, also known as etonogestrel), and norethindrone acetate (which is converted in vivo to norethindrone).

In some embodiments, the progestin is desogestrel, etonogestrel, norgestimate, or combinations thereof.

In some embodiments, the active agent is a modified testosterone, e.g., a derivative of the synthetic steroid ethisterone. In some embodiments, the modified testosterone is danazol.

In some embodiments, the intravaginal device of the present invention contains two active agents, such as a progestin and an estrogen. In some embodiments, the intravaginal device contains etonogestrel and ethinyl estradiol. In some embodiments, a combination of a progestin and an estrogen suitable for use with the present invention can be useful for contraceptive regimens.

In some embodiments, two or more active agents are dispersed together in the matrix. In some embodiments, two or more active agents are dispersed in separate compartments in the matrix.

As used herein, an “anesthetic” is a compound that blocks the passage of pain impulses in nerve pathways to the brain and induces a loss of sensation in one or more areas of the body without loss of vital functions. Anesthetics for use with the present invention include, but are not limited to, lidocaine, articaine, benoxinate, bupivacaine, dibucaine, mepivicaine, naepaine, piperocaine, procaine, prilocalne, tetracaine, and combinations thereof. In some embodiments, the anesthetic is lidocaine.

As used herein, an “anticholinergic” is a compound that blocks the neurotransmitter acetylcholine in the central and the peripheral nervous system. Anticholinergics for use with the present invention include, but are not limited to, oxybutynin, bethanechol, propiverine, propantheline, methylbenactyzium, scopolamine, tolterodine, trospium, combinations thereof, and salts thereof. In some embodiments, the anticholinergic is oxybutynin or a salt thereof. In some embodiments, the anticholinergic is oxybutynin hydrochloride.

In some embodiments, the intravaginal device of the present invention further comprises an excipient. As used herein, an “excipient” refers to a substance that is used in the formulation of pharmaceutical compositions, and, by itself, generally has little or no therapeutic value. One of skill in the art will recognize that a wide variety of pharmaceutically acceptable excipients can be used with the present invention including those listed in the Handbook of Pharmaceutical Excipients, Pharmaceutical Press 4th Ed. (2003) and Remington: The Science and Practice of Pharmacy, Lippincott Williams & Wilkins, 21st Ed. (2005), which are incorporated herein by reference in their entirety. As used herein, the term “pharmaceutically acceptable” refers to those compounds, materials, and/or compositions which are, within the scope of sound medical judgment, suitable for contact with the tissues of human beings and animals without excessive toxicity, irritation, allergic response, or other possible complications commensurate with a reasonable benefit/risk ratio.

In some embodiments, the matrix of the intravaginal device of the present invention determines or controls the rate of release of an active agent contained therein. As used herein, the “rate of release” or “release rate” refers to an amount or concentration of active agent that is released from the device over a defined period of time.

The polymer material can be used to control the rate of release of an active agent from the intravaginal device of the present invention. For example, the rate of release of an active agent from the device can be controlled by controlling the degree of cross-linking present in the polymer matrix. A high degree of cross-linking would be expected to result in a lower rate of release of the active agent from the polymer matrix. The degree of crosslinking can be controlled by the amount of crosslinker or catalyst used during production of the intravaginal device. See, e.g., U.S. Pat. No. 6,394,094. In some embodiments, permeation enhancers can be added to the matrix to increase the rate of release of the active agent from the matrix.

The rate of release of an active agent can also be controlled by the location of the active agent in the intravaginal device. For example, in the “core-and-sheath-type” intravaginal device, the location of the active agent—the core or the sheath—can modulate or control the rate of release of the active agent. In these embodiments, the active agent in the core of the vaginal device can have a lower rate of release into the vaginal tract than the active agent in the sheath, because the active agent in the core must diffuse through both the core and outer sheath before it reaches the vaginal tract, whereas the active agent in the outer sheath must only diffuse through the outer sheath before reaching the vaginal tract. In some embodiments, the device of the present invention further comprises an outer sheath. The outer sheath can control the rate of release of the active agent.

The rate of release of an active agent can also be controlled by pretreating the active agent. For example, in those embodiments of the invention in which the active agent is a steroid hormone in micronized form, the micronized steroid can be pretreated with an additional agent, such as ethyl cellulose, which coats the micronized steroid hormone particles. When the pretreated micronized steroid particles are incorporated into the polymer matrix during production of the intravaginal device (e.g., a vaginal ring), the rate of release of the hormone is slowed down or reduced compared to the rate of untreated micronized steroid.

The rate of release of an active agent can also be controlled or modulated by the addition to the polymer matrix of additional agents or excipients, such as, for example, mineral oil, or fatty acid esters.

The amount of active agent released from the device can be determined by a qualified healthcare professional and is dependent on many factors, e.g., the active agent, the condition to be treated, the age and/or weight of the subject to be treated, etc. In some embodiments, the active agent is released from the device at an average rate of about 0.01 mg to about 10 mg per 24 hours in situ, or about 0.05 mg to about 5 mg per 24 hours in situ, or about 0.1 mg to about 1 mg per 24 hours in situ. In some embodiments, the active agent is released from the device at an average rate of about 1 mg to about 100 mg per 24 hours in situ or about 5 mg to about 50 mg per 24 hours in situ.

In some embodiments, two or more active agents can be released from the device at a different rate per 24 hours in situ. For example, an estrogen can be released from the device at an average rate of about 0.01 mg to about 0.1 mg per 24 hours in situ and a progestin can be released from the device at an average rate of about 0.08 mg to about 0.2 mg per 24 hours in situ, or an estrogen can be released from the device at an average rate of about 0.1 mg to about 1 mg per 24 hours in situ and a progestin can be released from the device at an average rate of about 0.05 mg to about 5 mg per 24 hours in situ, or an estrogen can be released from the device at an average rate of about 0.05 mg to about 5 mg per 24 hours in situ and a progestin can be released from the device at an average rate of about 1 mg to about 100 mg per 24 hours in situ.

The release rate can be measured in vitro using, e.g., the USP Apparatus Paddle 2 method. The device is placed into a 500 ml solution of 0.05 M SDS at 37° C. with a paddle speed of 50 rpm. The active agent can be assayed by methods known in the art, e.g., by HPLC.

In some embodiments of the present invention, an active agent is released from the intravaginal device at a steady rate for up to about 1 month or about 30 days after administration to a female, for up to about 25 days after administration to a female, for up to about 21 days after administration to a female, for up to about 15 days after administration to a female, for up to about 10 days after administration to a female, for up to about 7 days after administration to a female, or for up to about 4 days after administration to a female.

As used herein, a “steady rate” is a release rate that does not vary by an amount greater than 70% of the amount of active agent released per 24 hours in situ, by an amount greater than 60% of the amount of active agent released per 24 hours in situ, by an amount greater than 50% of the amount of active agent released per 24 hours in situ, by an amount greater than 40% of the amount of active agent released per 24 hours in situ, by an amount greater than 30% of the amount of active agent released per 24 hours in situ, by an amount greater than 20% of the amount of active agent released per 24 hours in situ, by an amount greater than 10% of the amount of active agent released per 24 hours in situ, or by an amount greater than 5% of the amount of active agent released per 24 hours in situ.

In some embodiments, the active agent is a progestin with a steady release rate of active agent in situ of about 80 μg to about 200 μg per 24 hours, about 90 μg to about 150 μg per 24 hours, about 90 μg to about 125 μg per 24 hours, or about 95 μg to about 120 μg per 24 hours.

In some embodiments, the active agent is an estrogen with a steady release rate of active agent in situ of about 10 μg to about 100 μg per 24 hours, about 10 μg to about 80 μg per 24 hours, about 10 μg to about 60 μg per 24 hours, about 10 μg to about 40 μg per 24 hours, about 10 μg to about 20 μg per 24 hours, or about 10 μg to about 15 μg per 24 hours.

In some embodiments, various concentrations of active agent are present in the intravaginal device of the present invention. In some embodiments, an active agent is supersaturated in the matrix at 25° C. As used herein, “supersaturated” can refer to the amount of an active agent that is about one to about ten times the amount necessary to obtain the saturation concentration of the active agent in the matrix at 25° C.

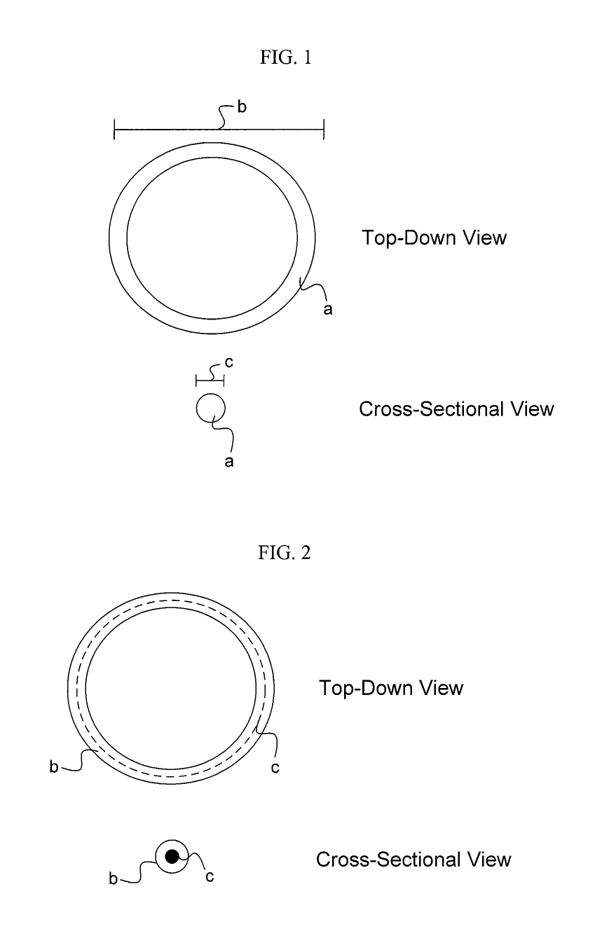

The device of the present invention can be any size suitable for placement in a vaginal tract. In some embodiments, the device has an outer diameter of about 40 mm to about 70 mm, about 45 mm to about 65 mm, or about 50 mm to about 60 mm. As used herein, an “outer diameter” refers to any straight line segment that passes through the center of the device and whose endpoints are on the outer perimeter of the device, see, e.g., FIG. 1, part b. In some embodiments, the outer diameter is the longest straight line segment that passes through the center of the device and whose endpoints are on the outer perimeter of the device.

In some embodiments, the device has an inner diameter of about 10 mm to about 60 mm, about 10 mm to about 50 mm, about 10 mm to about 40 mm, about 20 mm to about 40 mm, about 10 mm to about 30 mm, or about 10 mm to about 20 mm. As used herein, an “inner diameter” refers to any straight line segment that passes through the center of the device and whose endpoints are on the inner perimeter of the device. In some embodiments, the inner diameter is the longest straight line segment that passes through the center of the device and whose endpoints are on the inner perimeter of the device.

Intravaginal devices described previously had cross-sectional diameters of about 5 mm to about 10 mm. See, e.g., U.S. Pat. No. 4,822,616. The size of the intravaginal device of the present invention can incorporate the sizes described previously. Additionally, the intravaginal device of the present invention can have a smaller cross-sectional diameter. As used herein, a “cross-sectional diameter” refers to the longest straight line segment that passes through the center of the device and whose endpoints are on the outer perimeter of the device, see, e.g., FIG. 1, part c. In some embodiments, the cross-sectional diameter refers to the longest straight line segment that passes through the center of the support and whose endpoints are on the outer perimeter of the support. In some embodiments, the device has a cross-sectional diameter of about 1 mm to about 10 mm, about 1 mm to about 8 mm, about 2 mm to about 7 mm, about 3 mm to about 7 mm, about 4 mm to about 6.5 mm, about 5 mm to about 6 mm, or about 6 mm. In some embodiments, the device has a cross-sectional diameter of about 1 mm to about 6 mm, about 2 mm to about 4 mm, about 3 mm to about 5 mm, or about 4 mm to about 6 mm.

In some embodiments, the support has a cross-sectional diameter of about 0.5 mm to about 4 mm, about 0.5 mm to about 3.5 mm, about 0.5 mm to about 3 mm, about 1 mm to about 4 mm, about 1.5 mm to about 4 mm, or about 2 mm to about 3.5 mm.

In some cases, intravaginal devices without an additional support have reduced physical integrity relative to intravaginal devices with a support. Reducing the cross-sectional diameter of intravaginal devices without a support further reduces the physical integrity of the device, thus making its use impractical. Therefore, in some embodiments, the present invention is directed to an intravaginal device having a reduced cross-sectional diameter but having a rigid support, the device having equal or greater physical integrity relative to a device lacking a support.

The intravaginal device of the present invention can be in various shapes and sizes, thereby producing a device with various surface areas. As used herein, “surface area” refers to the total area of the device that is exposed to the environment, e.g., to the vagina, cervix, or uterus of a female. In some embodiments, the device has a surface area of about 800 mm² to about 2000 mm², about 1000 mm² to about 2000 mm², about 1200 mm² to about 2000 mm², about 1400 mm² to about 2000 mm², or about 1600 mm² to about 2000 mm².

### Methods of Making the Intravaginal Device

Various methods can be used to make the intravaginal devices of the present invention. Various means of producing intravaginal devices without rigid supports are known in the art. See, e.g., U.S. Pat. Nos. 6,544,546; 6,394,094; and 4,155,991. In some embodiments, the present invention is directed to a method of making an intravaginal device by molding the matrix of the device around the support. In some embodiments, this method comprises (i) placing the support in a mold, wherein the support has a Shore A Hardness of at least about 20 and a tensile strength of at least about 1 MPa, and (ii) adding the matrix to the mold, wherein the matrix has an active agent dispersed therein, wherein the support and the matrix are adjacent and wherein the device is annular.

The present invention is also directed to an intravaginal device made by this method. In some embodiments, the matrix is in a heated liquid state prior to being placed in the mold. In some embodiments, the heated liquid matrix solidifies upon cooling. In some embodiments, the matrix in a liquid state solidifies with the addition of a catalyst. In some embodiments, the matrix is formed first, and then the rigid support is attached to the matrix, e.g., by inserting the support into the matrix or attaching it to the outside of the matrix. In some embodiments, an adhesive is used to attach the rigid support to the matrix.

In some embodiments, compression molding is used to form the device of the present invention. In some embodiments, compressing a substantially homogeneous mixture to form a compressed matrix around a rigid support can be achieved by compression molding, or alternatively, by the use of a die press. As used herein, “compressed” refers to a mixture that has been compacted or fused under pressure. A compressed mixture has a density that is greater than the mixture prior to compression.

The substantially homogeneous mixtures for use with the present invention can be prepared using a wide variety of methods for mixing the ingredients, including any method recognized by artisans skilled in the methods of mixing ingredients, that results in a substantially homogeneous mixture suitable for compressing. Such methods include, but are not limited to, the following:

Dry powder blending: dry ingredients, except for the lubricant, if one is present, are combined and mixed using a suitable low shear diffusion-type mixer, or other mixing device, for a period of time sufficient to result in a substantially homogeneous dry mixture. In some embodiments, a lubricant is added after an initial period of mixing, which can be followed by remixing, at least until a second substantially homogeneous dry mixture is formed, which can be compressed to form a compressed intravaginal device of the present invention using, for example, a compression press.

Alternatively, the dry ingredients, except for the lubricant, if one is present, are combined sequentially and mixed for a sufficient period of time after each ingredient is sequentially added to the mixture, to achieve a substantially homogeneous mixing of the ingredients. Sequential mixing of the ingredients can be followed by compressing the substantially homogeneous mixture to form a compressed intravaginal device using, for example, a CARVER® Laboratory Press (Fred S. Carver, Inc., New York, N.Y.). In some embodiments, sequential mixing comprises geometric dilution.

Wet granulation: an active agent, a bulking agent, and other ingredients are dissolved or suspended in a liquid medium, and mixed using a high shear mixing apparatus until a substantially homogeneous paste is formed. The substantially homogeneous paste can then be dried, ground, and sized to form a substantially homogeneous dry granulation or powder, which can then be compressed to form a compressed intravaginal device of the present invention using methods and equipment known to those skilled in the art of dry powder compression.

Not being bound by any particular theory, the process of mixing can be optimized based on the plastic and elastic properties of the mixture. The plastic and elastic properties of the mixture are substantially inversely related to one another. Thus, as the plasticity of a mixture increases, its elasticity decreases. During compression, the plasticity of the substantially homogeneous mixture permits the mixture to retain the shape that is formed by compressing the mixture. The components of a compressed intravaginal device can be selected to allow the device to be fabricated using a rapid, cost-efficient compression process.

In some embodiments, the method of the present invention further comprises curing the compressed intravaginal device. As used herein, “curing” refers to a process useful to solidify, harden, or cross-link a substantially homogeneous compressed composition of the present invention. Curing can comprise heating, drying, crystallizing, cross-linking, photo-curing (e.g., exposing to monochromatic or broad-band ultraviolet, visible, or infrared light) or combinations thereof.

### Methods of Treatment

The intravaginal devices can be used for treating various conditions. The present invention can be directed to methods of contraception, the method comprising administering to a female the intravaginal device of the present invention.

The present invention can be directed to methods of hormone replacement therapy, the method comprising administering to a female the intravaginal device of the present invention.

As used herein, “female” refers to any animal classified as a mammal, including humans and non-humans, such as, but not limited to, domestic and farm animals, zoo animals, sports animals, and pets. In some embodiments, female refers to a human female.

As used herein, the term “administering to” refers to placing an intravaginal device of the present invention in contact with the vaginal and/or urogenital tract of a female.

The terms “treat” and “treatment” refer to both therapeutic treatment and prophylactic, maintenance, or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms or signs; diminishment of extent of condition, disorder or disease; stabilization (i.e., not worsening) of the state of condition, disorder or disease; delay in onset or slowing of condition, disorder or disease progression; amelioration of the condition, disorder or disease state, remission (whether partial or total), whether detectable or undetectable; or enhancement or improvement of condition, disorder or disease. Treatment includes eliciting a clinically significant response, without excessive levels of side effects. Treatment also includes prolonging survival as compared to expected survival if not receiving treatment.

In some embodiments, the present invention can be directed to a method of site specific drug delivery to the vaginal and/or urogenital tract, and the treatment of any disease in which the active agent can be absorbed in the vaginal and/or urogenital tract. In some embodiments, the intravaginal device of the present invention can be administered alone or in conjunction with other medications or pharmaceutical compositions.

An intravaginal device of the present invention contains a therapeutically effective amount of active agent. The term “therapeutically effective amount” refers to an amount of active agent that diminishes one or more symptoms of a disease or disorder (i.e., treats a disease or disorder) in a subject. The precise therapeutic dosage of an active agent necessary to be therapeutically effective can vary between subjects (e.g., due to age, body weight, sex, condition of the subject, the nature and severity of the disorder or disease to be treated, and the like). Thus, the therapeutically effective amount cannot be specified in advance and can be determined by a caregiver, for example, by a physician or other healthcare provider, using various means, for example, dose titration. Appropriate therapeutically effective amounts can also be determined by routine experimentation using, for example, animal models.

In some embodiments, the intravaginal device can be used as a method of providing contraception to a female for the treatment of a condition or disorder, or as a method of providing contraception and treating a condition or disorder in a female. Such conditions and disorders include, but are not limited to: breakthrough bleeding; irregular withdrawal bleeding; menstrual bleeding disorders; symptoms associated with an ovarian cyst, uterine leiomyoma (fibroid tumor), and/or Polycystic Ovarian Syndrome; hirsutism; iron deficiency anemia; menstrual disorders; acne; endometriosis; endometrial cancer; ovarian cancer; benign breast disease; infections; ectopic pregnancy; temporomandibular disorder; catamenial symptoms; non-menstrual related headache, nausea, and/or depression; peri-menopausal symptoms; hypoestrogenism; menopausal disorders; and loss of bone density.

In some embodiments, an intravaginal device of the present invention can be administered to provide contraception and treat a condition or disorder in a female, wherein the female is in need of both contraception and treatment of the condition or disorder. The female can be, for example, of childbearing age or peri-menopausal.

As used herein, a “peri-menopausal female” refers to a woman who has not yet definitely arrived at menopause but who is experiencing symptoms associated with menopause. “Peri-menopause” means “about or around the time of menopause” and encompasses the years preceding the last menstrual period during which ovarian function declines and ultimately ceases and can include the presence of symptoms and irregular cycles. As used herein, a “menopausal female” refers to a woman who has definitely arrived at menopause and may be experiencing symptoms associated with menopause. Menopause or post-menopause is the permanent cessation of menstruation after the loss of ovarian activity and is generally defined clinically as the absence of menstruation for about one year. Menopause may occur naturally in a woman or it may be artificially induced, e.g., through surgical or chemical means. For example, removal of the ovaries, which can occur, e.g., through hysterectomy, frequently leads to symptoms associated with menopause.

In some embodiments, an intravaginal device of the present invention can be administered to a subject to treat a menopausal condition. As used herein, a “menopausal condition” refers to a condition associated with menopause, or the period of natural cessation of menstruation. Additionally, the term “menopausal condition” can relate to a condition related to peri-menopause, post-menopause, or oophorectomized women, or women whose endogenous sex hormone production has been suppressed by a pharmaceutical chemical composition, e.g., a GnRH agonist such as leuprolide-acetate sold under the trademark LUPRONE® (TAP Pharmaceutical Products, Inc., Lake Forest, Ill.) or goserelin acetate, sold under the trademark ZOLADEX® (AstraZeneca Pharmaceuticals, Wilmington, Del.).

Various menopausal conditions are known in the art. Menopausal conditions include, but are not limited to, hot flashes, vaginal dryness, pain during intercourse, increased risk of infection, inability to control urination (e.g., urinary incontinence), increased frequency of urinary infection, vaginal atrophy, kraurosis vulvae, hot flashes and/or night sweats, fatigue, emotional changes (e.g., mood swings and changes in sexual interest), sleep disturbances (e.g., insomnia), dry skin and hair, increased growth of facial and body hair, increased risk of heart disease, aches and pains in the joints, headaches, palpitations (i.e., rapid, irregular heart beats), vaginal itching, osteoporosis, osteopenia, and generalized itching.

In some embodiments, an intravaginal device of the present invention can be administered to a subject to treat osteoporosis. As used herein, “osteoporosis” refers to a condition characterized by a decrease in bone mass and density, causing bones to become fragile. In some embodiments, osteoporotic conditions include increased risk of fracture, especially fractures of the hip or spine.

In some embodiments, an intravaginal device of the present invention can be administered to a subject to treat urinary incontinence. As used herein, “urinary incontinence” refers to the complete or partial loss of bladder control, resulting in frequent urination and/or uncontrolled urination.

In some embodiments, an intravaginal device of the present invention can be administered to a subject to treat vaginal infection. As used herein, “vaginal infection” refers to a bacterial or viral infection in or around the vagina, cervix, or uterus. Symptoms of vaginal infection include, but are not limited to, itching, burning, soreness, pain during intercourse and/or urination, and can be accompanied by vaginal discharge.

In some embodiments, an intravaginal device of the present invention can be administered to a subject to treat vaginal pain. As used herein, “vaginal pain” refers to pain localized in the female reproductive tract, e.g., the vagina, cervix, or uterus, and combinations thereof. The pain can be due to a medical condition and/or psychological difficulties. Medical conditions can include chronic diseases, minor ailments, and medications. Psychological causes can be related to physical or sexual abuse. As used herein, “abdominal pain” refers to pain in the region of the stomach, small intestine, large intestine, or bowel.

In some embodiments, an intravaginal device of the present invention can be administered to a subject to treat inflammation. As used herein, “inflammation” refers to the body's natural response to injury or infection, in which the site of injury or infection might display various degrees of pain, swelling, heat, redness and/or loss of function.

### EXAMPLES

### Example 1

The support of an intravaginal ring of the present invention is prepared by forming medical grade high density polyethylene ((HDPE), MEDPOR® Biomaterial, Porex, Newnan, Ga.) into a ring shape. The HDPE is transferred to a Grieve oven (Grieve Corp., Round Lake, Ill.) for curing at about 90° C. for about six hours. The HDPE is then allowed to cool to room temperature, providing an HDPE rigid support. The HDPE is then placed in a mold.

The matrix of an intravaginal ring of the present invention is prepared by forming a homogeneous mixture of an estrogen, a progestin, and a silicone polymer and placing the ingredients in a Ross DPM-4 mixer (Ross double planetary mixer and dispenser supplied by Charles Ross & Son, Hauppauge, N.Y.), where the ingredients are mixed and degassed under vacuum for about 30 minutes or until the estrogen and progestin are distributed substantially homogeneously throughout the polymer matrix. This mixture is then mixed with a catalyst, e.g., methyl chloride, to start the polymerization process. The catalyzed mixture is added to the mold containing the HDPE rigid support, the mixture encompassing the mold. The mixture is then cured, forming an intravaginal ring having a silicone matrix encompassing an HDPE rigid support.

The mold is disassembled to remove the intravaginal ring and the ring is packaged in a heat sealed foil pouch.

The process yields an intravaginal ring with an outer diameter of about 40 mm to about 60 mm, an inner diameter of about 10 mm to about 40 mm, and a cross-sectional diameter of about 5 mm to about 8 mm. The support of the intravaginal ring has a cross-sectional diameter of about 2 mm to about 4 mm.

### CONCLUSION

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. An intravaginal device comprising:
(a) a rigid support having a Shore A Hardness of at least about 20 and a tensile strength of at least about 1 MPa;
(b) a matrix; and
(c) an active agent dispersed in the matrix;
wherein the support and the matrix are adjacent and wherein the device is annular.

2. The device of claim 1, wherein the support has a Shore A Hardness of about 20 to about 80.

3. The device of claim 1, wherein the support has a tensile strength of about 1 MPa to about 100 MPa.

4. The device of claim 1, wherein the support has a Shore A Hardness of about 20 to about 80 and a tensile strength of about 1 MPa to about 100 MPa.

5. The device of claim 1, wherein the support comprises a metal, an alloy, a plastic, composites thereof, or combinations thereof.

6. The device of claim 5, wherein the plastic comprises a thermoplastic or a thermoset plastic.

7. The device of claim 5, wherein the plastic comprises a polyalkylene, a polystyrene, a polysiloxane, a polyvinyl acetate, a polyvinyl chloride, a polyester, a polyurethane, an acrylic, a nylon, a dacron, a teflon, or combinations thereof.

8. The device of claim 7, wherein the polyalkylene comprises a high density polyethylene, a high density polypropylene, a high density polybutylene, or combinations thereof.

9. The device of claim 7, wherein the polyalkylene comprises a high density polyethylene.

10. The device of claim 1, wherein the matrix comprises a polysiloxane, a polyalkylene, a polystyrene, a polyvinyl acetate, a polyvinyl chloride, a polyester, a polyurethane, an acrylic, a nylon, a dacron, a teflon, or combinations thereof.

11. The device of claim 1, wherein the matrix is a polysiloxane, an ethylene-vinylacetate copolymer, or combinations thereof.

12. The device of claim 1, wherein the matrix encompasses at least 95% of the surface area of the support.

13. The device of claim 1, wherein the device comprises more than one matrix.

14. The device of claim 1, wherein the active agent is a steroid hormone, an anticholinergic, an anesthetic, combinations thereof, or derivatives thereof.

15. The device of claim 1, wherein the active agent is progesterone, estrogen, oxybutynin, lidocaine, danazol, etonogestrel, ethinyl estradiol, or combinations thereof.

16. The device of claim 1, wherein the device is a vaginal ring.

17. The device of claim 1, wherein the device has an outer diameter of about 40 mm to about 70 mm.

18. The device of claim 1, wherein the device has an inner diameter of about 10 mm to about 60 mm.

19. The device of claim 1, wherein the support has a cross-sectional diameter of about 0.5 mm to about 4 mm.

20. The device of claim 1, further comprising an outer sheath.

21. A method of making an intravaginal device having a rigid support and a matrix, the method comprising:
(i) placing the support in a mold, wherein the support has a Shore A Hardness of at least about 20 and a tensile strength of at least about 1 MPa; and
(ii) adding the matrix to the mold, wherein the matrix has an active agent dispersed therein;
wherein the support and the matrix are adjacent and wherein the device is annular.

22. An intravaginal device made by the method of claim 21.

23. A method of contraception, the method comprising administering to a female the device of claim 14.

24. A method of hormone replacement therapy, the method comprising administering to a female the device of claim 14.
